(19) United States
(12) Patent Application Publication
(10) Pub. No.: US 2024/0180554 A1
(43) Pub. Date: Jun. 6, 2024

(54) SURGICAL INSTRUMENT INCLUDING A DECOUPLING MECHANISM

(71) Applicant: Covidien LP, Mansfield, MA (US)

(72) Inventors: Jacob C. Baril, Norwalk, CT (US); Justin J. Thomas, New Haven, CT (US); Saumya Banerjee, Southington, CT (US)

(21) Appl. No.: 18/269,340

(22) PCT Filed: Dec. 10, 2021
(86) PCT No.: PCT/US2021/062802

§ 371 Date: Jun. 23, 2023

Related U.S. Application Data
(60) Provisional application No. 63/129,784, filed on Dec. 23, 2020.

(51) Int. Cl. A61B 17/072; A61B 17/00; A61B 17/29

(52) U.S. Cl. CPC A61B 17/07207; A61B 2017/00477; A61B 2017/2903

(57) ABSTRACT

A surgical instrument includes a decoupling mechanism and a loading unit. The decoupling mechanism includes a driver coupling, an overstroke sled, a proximal driver, and a distal driver. The overstroke sled includes bosses configured to slidably engage slots of the driver coupling. The distal driver is coupled to a housing of the driver coupling for concomitant rotation therewith. A drive assembly of the loading unit is coupled to the distal driver such that rotation of the distal driver causes axial displacement of the drive assembly. The decoupling mechanism is transitionable between a first state, in which, rotation of the proximal driver of the decoupling mechanism rotates the driver coupling and the distal driver, thereby advancing the drive assembly, and a second state, in which, rotation of the proximal driver disengages the overstroke sled from the driver coupling to enable rotation of the overstroke sled relative to the driver coupling.

### CROSS REFERENCE TO RELATED APPLICATIONS

This application claims benefit of U.S. Provisional Patent Application No. 63/129,784, filed Dec. 23, 2020, the entire contents of each of which are incorporated by reference herein.

### BACKGROUND

### Technical Field

The disclosure relates to surgical instruments and, more particularly, to a surgical instrument including a decoupling mechanism that operatively decouples an actuator from a drive assembly of a surgical loading unit.

### Background of Related Art

Electromechanical surgical instruments include a reusable handle assembly and disposable loading units and/or single use loading units. The loading units are selectively connected to the handle assembly prior to use and disconnected from the handle assembly following use in order to be disposed of or in some instances sterilized for re-use. Various loading units have different requirements such as, e.g., axial displacement or torque.

Accordingly, there is a need for a surgical instrument that can accommodate various surgical loading units without compromising the performance requirements, while overcoming compatibility challenges associated with utilizing a plurality of surgical loading units having different requirements such as, e.g., torque or axial displacement.

### SUMMARY

In accordance with the disclosure, a surgical instrument includes a decoupling mechanism and a loading unit. The decoupling mechanism includes a driver coupling, an overstroke sled, a proximal driver, and a distal driver. The driver coupling has a housing defining a bore, an aperture in communication with the bore, and slots defined at one end of the bore. The overstroke sled includes bosses configured to be slidably received within the slots of the driver coupling. The proximal driver is rotatably coupled to an actuator of the surgical instrument and threadably coupled to the overstroke sled. The distal driver is coupled to the housing of the driver coupling for concomitant rotation therewith. The loading unit includes a drive assembly configured for axial displacement along a length of the loading unit. The drive assembly is coupled to the distal driver such that rotation of the distal driver of the decoupling mechanism causes axial displacement of the drive assembly. The decoupling mechanism is transitionable between a first state, in which, rotation of the proximal driver of the decoupling mechanism rotates the driver coupling and the distal driver, thereby advancing the drive assembly, and a second state, in which, rotation of the proximal driver disengages the overstroke sled from the driver coupling to enable rotation of the overstroke sled relative to the driver coupling.

In an aspect, the bosses of the overstroke sled may be configured to be received within the slots of the driver coupling such that the overstroke sled imparts rotation to the driver coupling.

In another aspect, the driver coupling may include a spring in the bore of the driver coupling positioned to bias the overstroke sled towards the slots of the driver coupling.

In yet another aspect, the loading unit may include a knife assembly and an actuation sled coupled to the drive assembly of the loading unit for axial displacement with the drive assembly.

In still yet another aspect, the distal driver may be threadably coupled to the drive assembly of the loading unit such that rotation of the distal driver causes axial displacement of the drive assembly.

In still yet another aspect, the overstroke sled may include a cylindrical body having an outer surface. The bosses may be diametrically disposed on the outer surface of the cylindrical body.

In an aspect, the slots of the driver coupling may be diametrically arranged and extend along a portion of a length of the driver coupling.

In another aspect, the slots of the driver coupling may be disposed adjacent the aperture of the driver coupling. The aperture may be dimensioned to receive the proximal driver.

In yet another aspect, the proximal driver, the overstroke sled, and the driver coupling may be concentrically arranged.

In an aspect, the proximal and distal drivers may be axially aligned.

In accordance with another aspect of the disclosure, a surgical instrument includes a shaft assembly, a decoupling mechanism, and a loading unit. The shaft assembly includes a tubular shaft defining a lumen therethrough. The tubular shaft defines a slot. The decoupling mechanism is movably disposed within the lumen of the tubular shaft and includes a driver coupling, an overstroke sled, a proximal driver, and a distal driver. The driver coupling has a housing defining a bore, an aperture in communication with the bore, and slots. The housing includes a tab on an outer surface thereof. The overstroke sled includes bosses configured to slidably engage the slots of the driver coupling. The proximal driver is threadably coupled to the overstroke sled. The distal driver is coupled to the housing of the driver coupling for concomitant axial displacement therewith. The loading unit includes a drive assembly configured for axial displacement along a length of the loading unit. The drive assembly is coupled to the distal driver of the decoupling mechanism such that the distal driver imparts axial displacement to the drive assembly. The decoupling mechanism is transitionable between a first state, in which, rotation of the proximal driver of the decoupling mechanism advances the overstroke sled with the driver coupling, thereby advancing the drive assembly, and a second state, in which, rotation of the proximal driver disengages the overstroke sled from the driver coupling to enable rotation of the overstroke sled relative to the driver coupling.

In an aspect, the driver coupling may include a spring in the bore of the driver coupling positioned to bias the overstroke sled towards the slots of the driver coupling.

In another aspect, the loading unit may include a knife assembly and an actuation sled coupled to the drive assembly of the loading unit for axial displacement with the drive assembly.

In yet another aspect, the overstroke sled may include a cylindrical body with the bosses diametrically disposed on an outer surface of the cylindrical body.

In still yet another aspect, the slots of the driver coupling may be diametrically arranged and extend along a portion of a length of the driver coupling.

In still yet another aspect, the tab of the driver coupling may extend axially along a portion of a length of the driver coupling.

In still yet another aspect, the proximal driver, the overstroke sled, and the driver coupling may be concentrically arranged.

In still yet another aspect, the proximal and distal drivers may be axially aligned.

In still yet another aspect, the bosses of the overstroke sled may be aligned with the tab of the driver coupling.

In still yet another aspect, when the drive assembly is in a distal position, rotation of the proximal driver may advance the bosses of the overstroke sled through the slots of the driver coupling.

### BRIEF DESCRIPTION OF THE DRAWINGS

Various aspects of the disclosure are described hereinbelow with reference to the drawings, which are incorporated and constitute a part of this specification, wherein:

FIG. 1 is a perspective view of a surgical instrument including a decoupling mechanism in accordance with the disclosure;

FIG. 2 is an enlarged perspective view of the indicated area of detail of FIG. 1;

FIG. 3 is a perspective view of the decoupling mechanism of FIG. 2 with a portion of a housing of a driver coupling of the decoupling mechanism removed;

FIG. 4 is an exploded perspective view of the decoupling mechanism of FIG. 2 with parts separated;

FIG. 5 is a perspective view of the knife assembly and the actuation sled of the surgical loading unit of FIG. 1;

FIG. 6 is a side cross-sectional view of the surgical loading unit of the surgical instrument of FIG. 1 taken along section line 6-6 of FIG. 1;

FIG. 7 is a cross-sectional view of a decoupling mechanism of FIG. 2 taken along section line 7-7 of FIG. 2;

FIG. 8 is a cross-sectional view of the decoupling mechanism of FIG. 1 taken along section line 8-8 of FIG. 7;

FIG. 9 is a side cross-sectional view of the surgical loading unit illustrating the knife assembly and the actuation sled in a distal-most position;

FIG. 10 is a side cross-sectional view of the decoupling mechanism of FIG. 2, illustrating activation of the decoupling mechanism;

FIG. 11 is a perspective view of another aspect of a decoupling mechanism for use with the surgical instrument of FIG. 1;

FIG. 12 is a cross-sectional view of the decoupling mechanism of FIG. 11 taken along section line 12-12 of FIG. 11;

FIG. 13 is a side cross-sectional view of the decoupling mechanism of FIG. 11 taken along section line 13-13 of FIG. 11; and

FIG. 14 is a side cross-sectional view of the decoupling mechanism of FIG. 13, illustrating activation of the decoupling mechanism.

### DETAILED DESCRIPTION

The disclosed surgical instrument is described in detail with reference to the drawings, in which like reference numerals designate identical or corresponding elements in each of the several views. As used herein, the term “distal,” as is conventional, will refer to that portion of the instrument, apparatus, device, or component thereof which is farther from the user, while the term “proximal” will refer to that portion of the instrument, apparatus, device, or component thereof which is closer to the user. As used herein, the terms parallel and perpendicular are understood to include relative configurations that are substantially parallel and substantially perpendicular up to about + or −10 degrees from true parallel and true perpendicular. In the following description, well-known functions or constructions are not described in detail to avoid obscuring the disclosure in unnecessary detail.

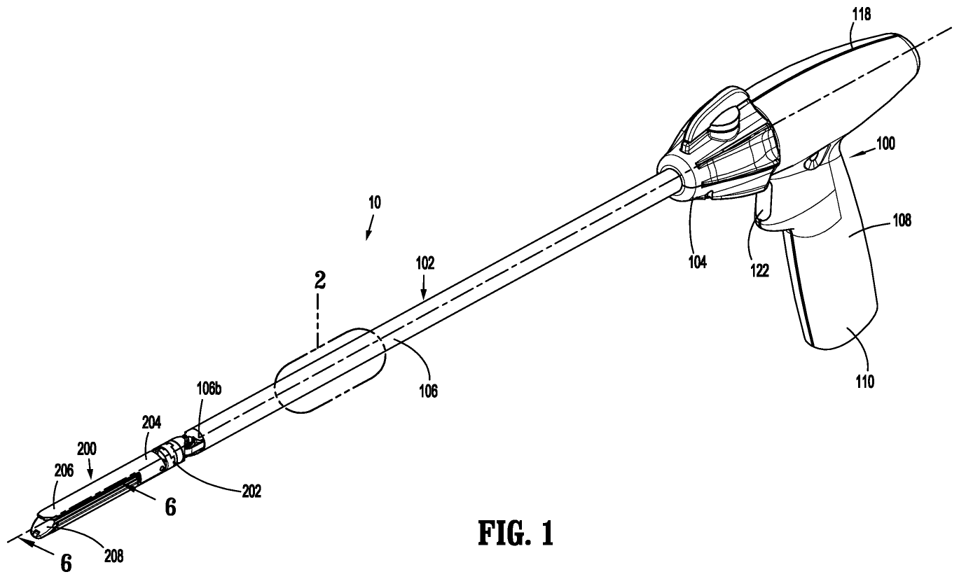

With reference to FIG. 1, a surgical instrument in accordance with the disclosure is generally designated as 10, and is in the form of a powered hand-held electromechanical surgical instrument configured for selective coupling of a plurality of different surgical loading units, for example, a surgical loading unit 200. The surgical loading unit 200 includes a proximal body portion 202 that is detachably coupled to a shaft assembly 102 of the surgical instrument 10, and an end effector 204. The proximal body portion 202 is releasably attached to a distal end portion 106b of an outer tube 106 of the shaft assembly 102. The end effector 204 includes a jaw member 206 having an anvil and a jaw member 208 including a cartridge assembly having a plurality of staples. The jaw member 208 is pivotable in relation to the jaw member 206 and is transitionable between an open or spaced apart configuration and a closed or approximated configuration.

The surgical instrument 10 further includes a handle assembly 100 and the shaft assembly 102. The shaft assembly 102 includes a knob housing 104 coupled to the handle assembly 100 and the outer tube 106 extending distally from the knob housing 104 and configured for selective connection with the surgical loading unit 200. It is contemplated that the handle assembly 100 includes a disposable and sterile handle housing 110 and an instrument module configured for removable receipt within handle housing 110. The handle housing 110 has a body such as, for example, a barrel portion 118, and a handle portion 108 extending perpendicularly downward from the barrel portion 118. The handle portion 108 may be configured for insertion or removal of the instrument module including a battery and a motor.

The surgical instrument module of the handle assembly 100 is configured for powering the surgical instrument 10. In other aspects, other means for powering surgical instrument 10 are contemplated, such as, for example, a battery-powered motor that is permanently fixed within handle housing 110. The instrument module may include a motor, such as, for example, an electrical drive motor, which is electrically connected or wirelessly connected to a printed circuit board and a battery. In aspects, the battery may include a boost circuit and may be rechargeable (e.g., wirelessly). The printed circuit board may include a USB charging connector to allow for the battery to be recharged with a USB charger or wirelessly (e.g., via induction). In aspects, the printed circuit board may have a motor controller or a processor.

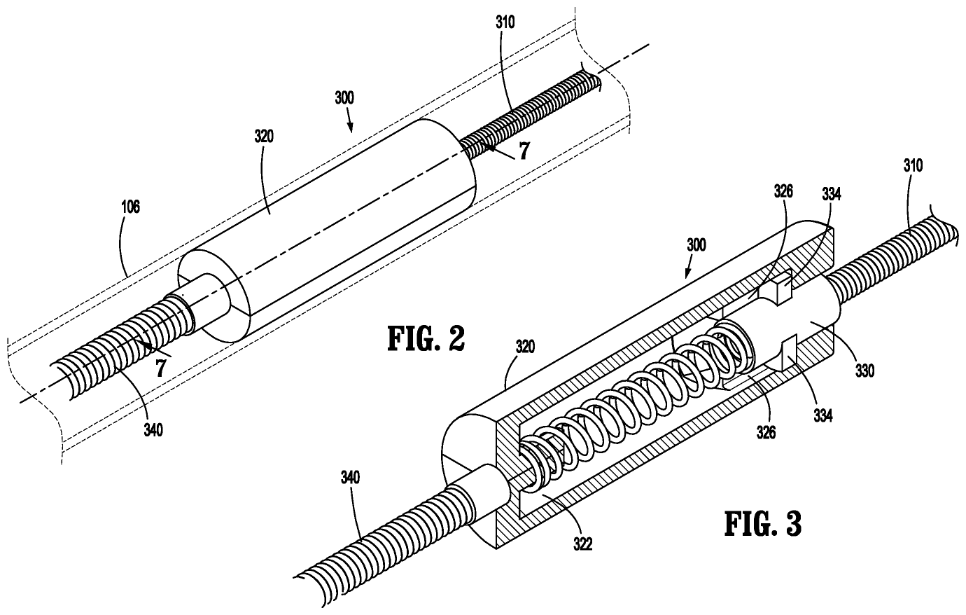

The handle assembly 100 has a fire switch 122 configured to actuate the surgical loading unit 200. Activation of the fire switch 122 activates an actuator such as, e.g., a motor, to impart rotation to a drive rod (not shown) of the shaft assembly. It is contemplated that the drive rod may be a flexible cable. The drive rod has a distal end portion configured to be coupled to a proximal driver 310 (FIG. 2) of the decoupling mechanism 300 that is operatively coupled to a drive assembly 250 (FIG. 6) of the surgical loading unit 200 to effect a function of the surgical loading unit 200. In this manner, rotation of the proximal driver 310 of the decoupling mechanism 300 advances the drive assembly 250 of the surgical loading unit 200, which closes the jaw members 206, 208 of the surgical loading unit 200 and fires the surgical loading unit 200.

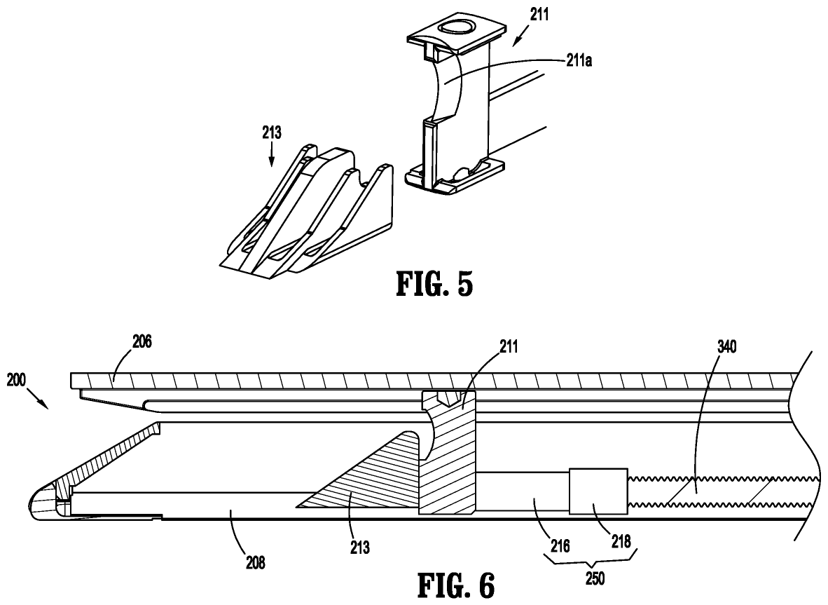

FIGS. 2 and 3 illustrate the decoupling mechanism 300 in accordance with the disclosure. The decoupling mechanism 300 is configured to operatively decouple the actuator of the handle assembly 100 (FIG. 1) from the surgical loading unit 200 (FIG. 1) once a threshold force or displacement of the drive assembly 250 (FIG. 6) of the surgical loading unit 200 is effected, thereby inhibiting damage to the components of the drive assembly 250 (FIG. 6) or other components of the surgical loading unit 200 due to excess rotation or torque. Under such a configuration, the surgical instrument 10 may be adapted for use with surgical loading units 200 having various operational requirements such as, e.g., axial displacement of the knife assembly 211 and the actuation sled 213 (FIG. 5) or torque requirements.

In aspects, the decoupling mechanism 300 is dimensioned to be disposed within the outer tube 106 of the shaft assembly 102 (FIG. 1). In particular, the decoupling mechanism 300 includes a proximal driver 310 operatively coupled to an actuator of the handle assembly 100 (FIG. 1), a driver coupling 320, an overstroke sled 330 threadably coupled to the proximal driver 310, and a distal driver 340 coupled to the driver coupling 320 for a concomitant rotation therewith.

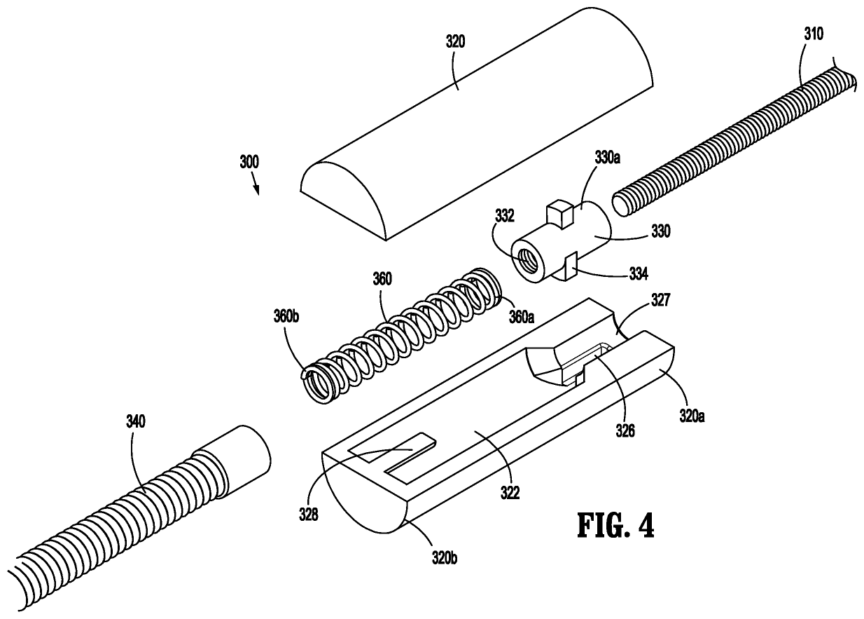

FIG. 4 illustrate the overstroke sled 330 having a cylindrical body 330a and defining a bore 332 threadably engageable with the proximal driver 310. The overstroke sled 330 includes bosses 334 that extend radially outwards from an outer surface of the cylindrical body 330a. The bosses 334 are configured to be slidably received in slots 326 of the driver coupling 320, as will be described below. The driver coupling 320 defines a bore 322 extending along a length of the driver coupling 320 and in alignment with the proximal and distal drivers 310, 340. In particular, the driver coupling 320 includes proximal and distal end portions 320a, 320b. The proximal end portion 320a defines an aperture 327 to receive the proximal driver 310 therethrough and the slots 326 are positioned adjacent the aperture 327. The slots 326 are in communication with the bore 322. Each slot 326 extends axially along a length of the driver coupling 320 and is dimensioned to receive the corresponding boss 334 of the overstroke sled 330 to inhibit relative rotation between the driver coupling 320 and the overstroke sled 330 when the bosses 334 of the overstroke sled 330 are received in the slots 326 of the driver coupling 320. Further, each slot 326 extends axially along a length of the driver coupling 320 to enable relative axial displacement therebetween. In addition, the proximal driver 310, the driver coupling 320, and the overstroke sled 330 may be concentrically arranged. The driver coupling 320 further includes a spring 360 disposed within the bore 322. A proximal end 360a of the spring 360 is secured to the overstroke sled 330 and a distal end 360b of the spring 360 is affixed to a distal end portion 320b of the driver coupling 320. For example, the driver coupling 320 may further include a tang 328 extending proximally from the distal end portion 320b of the driver coupling 320 into the bore 322. At least a portion of the spring 360 may be secured about the tang 328.

FIGS. 5 and 6 illustrate the surgical loading unit 200 in the form of a linear stapler as known in the art. The surgical loading unit 200 includes the drive assembly 250 configured to impart axial displacement to a knife assembly 211 including a knife edge 211a to cut tissue and an actuation sled 213 coupled to the knife assembly 211 for axial displacement with the knife assembly 211. The knife assembly 211 is operatively coupled to the jaw members 206, 208 such that when the knife assembly 211 is advanced, the jaw members 206, 208 are approximated to the closed configuration. Further, as the knife assembly 211 is advanced, the knife edge 211a of the knife assembly 211 cuts tissue. In addition, the actuation sled 213 sequentially ejects staples from a staple cartridge (not shown) of the jaw member 208 towards an anvil on the jaw member 206, as the knife assembly 211 is advanced. In particular, the drive assembly 250 includes an actuation rod 216 including an adapter portion 218 threadably engaging the distal driver 340 of the decoupling mechanism 300 such that rotation of the distal driver 340 causes axial displacement of the drive assembly 250.

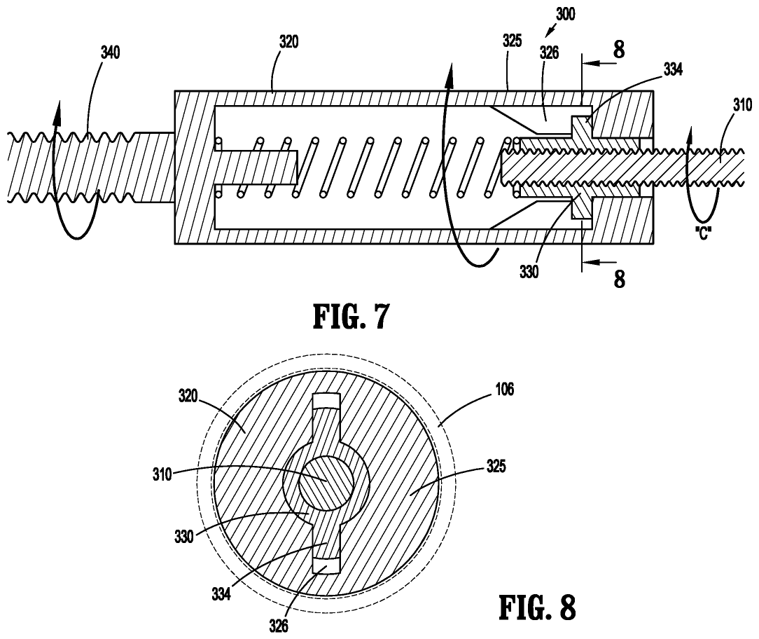

FIGS. 7 and 8 illustrate the operation of the decoupling assembly 300 during actuation of the surgical loading unit 200. When the fire switch 122 of the handle assembly 100 (FIG. 1) is pressed, the actuator such as, e.g., a motor, in the handle assembly 100 is activated and imparts rotation to the proximal driver 310 of the decoupling mechanism 300. Rotation of the proximal driver 310 rotates the overstroke sled 330. The bosses 334 of the overstroke sled 330 are received in the slots 326 of the driver coupling 320 such that rotation of the overstroke sled 330 imparts rotation to the driver coupling 320. In this manner, rotation of the driver coupling 320 imparts concomitant rotation to the distal drive 340, which, in turn, advances the drive assembly 250 (FIG. 6) of the surgical loading unit 200 to effect stapling and cutting of tissue through axial displacement of the knife assembly 211 and the actuation sled 213.

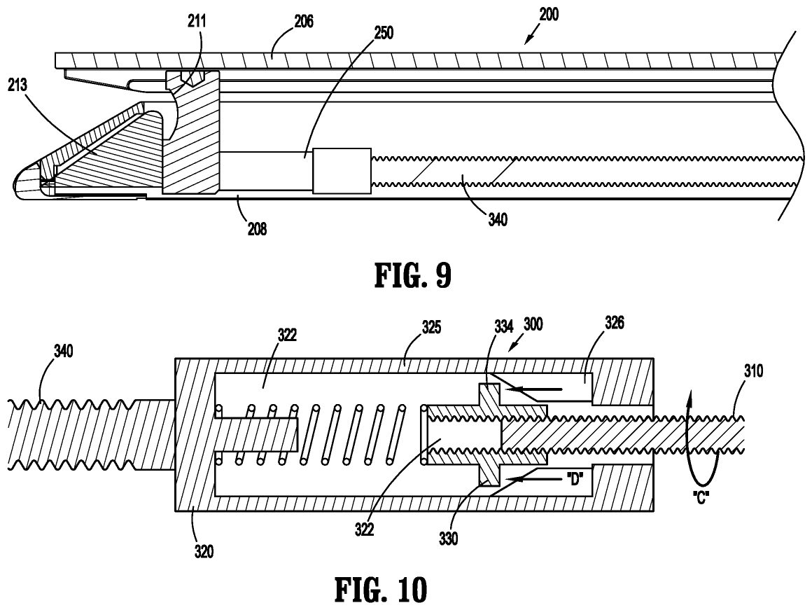

FIGS. 9 and 10 illustrate the knife assembly 211 and the actuation sled 213 advanced to a distal-most position. Under such a configuration, additional rotation of the proximal driver 310 does not impart rotation to the driver coupling 320. Rather, the overstroke sled 330 is axially displaced relative to the driver coupling 320. Specifically, when the proximal driver 310 is rotated, the bosses 334 of the overstroke sled 330 slides through the slots 326 of the driver coupling 320 in the direction of arrows “D” in FIG. 10 and the overstroke sled 330 operatively disengages from the driver coupling 320. In this manner, additional rotation of the proximal driver 310 causes rotation of the overstroke sled 330 in the bore 322 of the driver coupling 320 without imparting such rotation to the driver coupling 320. Decoupling of the overstroke sled 330 from the driver coupling 320 disengages the proximal driver 310 from the drive assembly 250, whereby additional rotation of the proximal driver 310 does not affect the components of the drive assembly 250 or other components of the surgical loading unit 200. In this manner, the rotational output of the actuator in the handle assembly 100 may be utilized across various surgical loading units having different requirements.

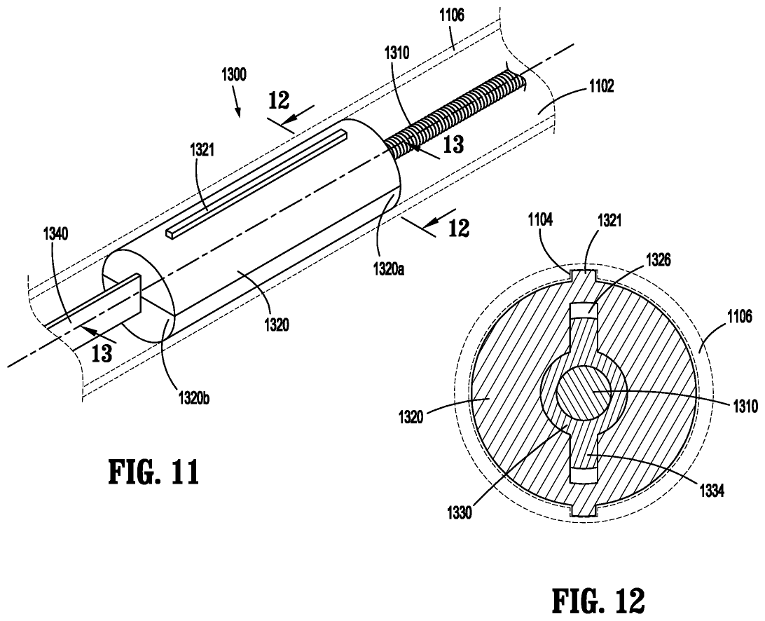

FIGS. 11 and 12 illustrate a decoupling mechanism 1300 in accordance with another aspect of the disclosure. Parts of the decoupling mechanism 1300 substantially identical to the parts of the decoupling mechanism 300 (FIG. 1) such as a motor, will only be described herein to the extent necessary to identify differences in construction and/or operation. The decoupling mechanism 1300 includes a proximal driver 1310 operatively coupled to an actuator of the handle assembly 100 (FIG. 1), a driver coupling 1320, an overstroke sled 1330 threadably coupled to the proximal driver 1310, and a distal driver 1340 coupled to the driver coupling 1320 for axial displacement therewith. In particular, the distal driver 1340 is coupled to the drive assembly 250 (FIG. 6) to impart axial displacement to the knife 211 and the actuation sled 213 (FIG. 9).

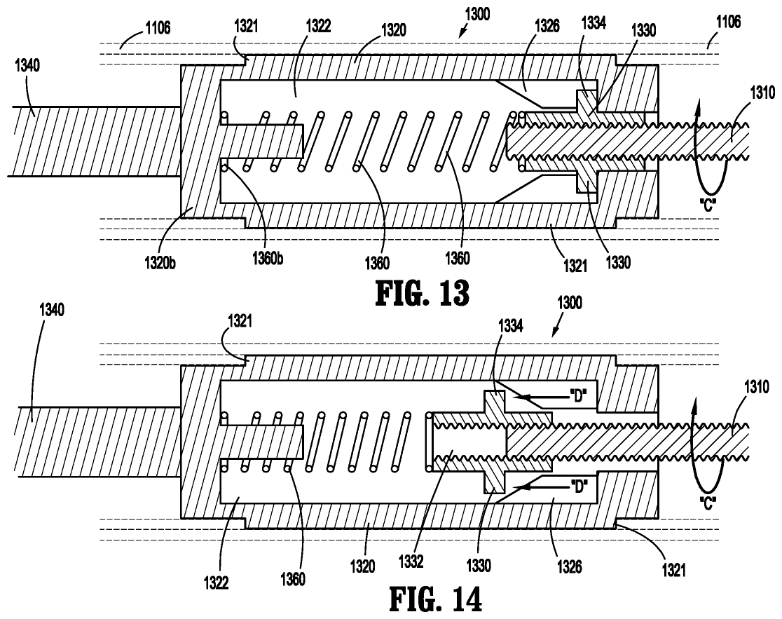

FIGS. 11 and 12 further illustrate, the overstroke sled 1330 defining a bore 1332 (FIG. 14) that is configured to threadably receive the proximal driver 1310. The overstroke sled 1330 includes bosses 1334 that diametrically oppose each other. The bosses 1334 extend laterally with respect to a length of the overstroke sled 1330. The bosses 1334 are configured to slidably engage slots 1326 of the driver coupling 1320. The driver coupling 1320 defines a bore 1322 (FIG. 13) extending along a length of the driver coupling 1320 and in alignment with the proximal and distal drivers 1310, 1340. In particular, the driver coupling 1320 includes proximal and distal end portions 1320a, 1320b. The proximal end portion 1320a defines slots 1326 in communication with the bore 1322. Each slot 1326 extends axially along a length of the driver coupling 1320 and is dimensioned to receive the boss 1334 of the overstroke sled 1330 such that bosses 1334 inhibit relative rotation between the driver coupling 1320 and the overstroke sled 1330 when the bosses 1334 are received in the slots 1326. Further, each slot 1326 extends axially along a length of the driver coupling 320 to enable relative axial displacement between the driver coupling 1320 and the overstroke sled 1330. The driver coupling 1320 further includes a spring 1360. A proximal end 1360a of the spring 1360 is secured to the overstroke sled 1330 and a distal end 1360b of the spring 1360 is affixed to a distal end portion 1320b of the driver coupling 1320. In contrast to the driver coupling 320 (FIG. 2) described hereinabove, the driver coupling 1320 includes a tab 1321 on an outer surface of thereof. In particular, the tab 1321 extends axially along a length of the driver coupling 1320. An outer tube 1106 of a shaft assembly 1102 defines a groove 1104 extending axially along a length of the outer tube 1106. The groove 1104 is configured to slidably receive the tab 1321 of the driver coupling 1320 to enable axial translation of the driver coupling 1320, while inhibiting relative rotation therewith.

FIGS. 13 and 14 illustrate the operation of the decoupling assembly 1300 during actuation of the surgical loading unit 200. When the fire switch 122 of the handle assembly 100 is pressed, the motor in the handle assembly 100 is activated and imparts rotation to the proximal driver 1310 of the decoupling mechanism 1300, which, in turn, advances the overstroke sled 1330. The bosses 1334 of the overstroke sled 1330 are received in the slots 1326 of the driver coupling 1320 such that axial displacement of the overstroke sled 1330 causes axial displacement of the driver coupling 1320. In this manner, axial displacement of the driver coupling 1320 imparts axial displacement to the distal driver 1340, which, in turn, advances the drive assembly 250 (FIG. 6) to effect stapling and cutting of tissue by the knife assembly 211 and the actuation sled 213.

FIG. 14 illustrates the driver coupling 1320 when the knife assembly 211 and the actuation sled 213 are in a distal-most position. Under such a configuration, the driver coupling 1320 is unable to advance further such that when the proximal driver 1310 is further rotated, the overstroke sled 1330 is axially displaced relative to the driver coupling 1320, i.e., the bosses 1334 of the overstroke sled 1330 slide through the slots 1326 in the direction of arrows “D”. In this manner, additional rotation of the proximal driver 1310 causes the overstroke sled 1330 now disengaged from the driver coupling 1320 to be rotated within the bore 1322 of the driver coupling 1320 relative to the driver coupling 1320. Decoupling of the overstroke sled 1330 from the driver coupling 1320 operatively disengages the proximal driver 1310 from the drive assembly 250 of the surgical loading unit 200, whereby additional rotation of the proximal driver 1310 does not affect the components of the drive assembly 250 or other components of the surgical loading unit 200, thereby minimizing any potential damage to the components. In this manner, the rotational output of the motor in the handle assembly 100 may be utilized in various surgical loading units having different requirements. The surgical instrument 10 may utilize the decoupling mechanism 300 or 1300. For example, a plurality of surgical loading units may be provided with each having a different length (e.g., 30 mm, 45 mm and 60 mm).

Any of the components described herein may be fabricated from either metals, plastics, resins, composites, or the like taking into consideration strength, durability, wearability, weight, resistance to corrosion, ease of manufacturing, cost of manufacturing, and the like. It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

It will be understood that various modifications may be made to the disclosed decoupling mechanism for use with surgical instruments. Therefore, the above description should not be construed as limiting, but merely as exemplifications of the disclosure. Those skilled in the art will envision other modifications within the scope and spirit of the disclosure.

1. A surgical instrument comprising:
a decoupling mechanism including:
a driver coupling having a housing defining a bore, an aperture in communication with the bore, and slots defined at one end of the bore;
an overstroke sled including bosses configured to be slidably received within the slots of the driver coupling;
a proximal driver rotatably coupled to an actuator of the surgical instrument, the proximal driver threadably coupled to the overstroke sled; and
a distal driver coupled to the housing of the driver coupling for concomitant rotation therewith; and
a loading unit including a drive assembly configured for axial displacement along a length of the loading unit, the drive assembly coupled to the distal driver of the decoupling mechanism such that rotation of the distal driver causes axial displacement of the drive assembly,
wherein the decoupling mechanism is transitionable between a first state, in which, rotation of the proximal driver of the decoupling mechanism rotates the driver coupling and the distal driver, thereby advancing the drive assembly, and a second state, in which, rotation of the proximal driver disengages the overstroke sled from the driver coupling to enable rotation of the overstroke sled relative to the driver coupling.

2. The surgical instrument according to claim 1, wherein the bosses of the overstroke sled are configured to be received within the slots of the driver coupling such that the overstroke sled imparts rotation to the driver coupling.

3. The surgical instrument according to claim 1, wherein the driver coupling includes a spring in the bore of the driver coupling positioned to bias the overstroke sled towards the slots of the driver coupling.

4. The surgical instrument according to claim 1, wherein the loading unit includes a knife assembly and an actuation sled coupled to the drive assembly of the loading unit for axial displacement with the drive assembly.

5. The surgical instrument according to claim 1, wherein the distal driver is threadably coupled to the drive assembly of the loading unit such that rotation of the distal driver causes axial displacement of the drive assembly.

6. The surgical instrument according to claim 1, wherein the overstroke sled includes a cylindrical body having an outer surface, the bosses diametrically disposed on the outer surface of the cylindrical body.

7. The surgical instrument according to claim 1, wherein the slots of the driver coupling are diametrically arranged and extend along a portion of a length of the driver coupling.

8. The surgical instrument according to claim 1, wherein the slots of the driver coupling are disposed adjacent the aperture of the driver coupling, the aperture dimensioned to receive the proximal driver.

9. The surgical instrument according to claim 1, wherein the proximal driver, the overstroke sled, and the driver coupling are concentrically arranged.

10. The surgical instrument according to claim 1, wherein the proximal and distal drivers are axially aligned.

11. A surgical instrument comprising:
a shaft assembly including a tubular shaft defining a lumen therethrough, the tubular shaft defining a slot;
a decoupling mechanism movably disposed within the lumen of the tubular shaft, the decoupling mechanism including:
a driver coupling having a housing defining a bore, an aperture in communication with the bore, and slots, the housing including a tab on an outer surface thereof;
an overstroke sled including bosses configured to slidably engage the slots of the driver coupling;
a proximal driver threadably coupled to the overstroke sled; and
a distal driver coupled to the housing of the driver coupling for concomitant axial displacement therewith; and
a loading unit including a drive assembly configure for axial displacement along a length of the loading unit, the drive assembly coupled to the distal driver such that the distal driver of the decoupling mechanism imparts axial displacement to the drive assembly,
wherein the decoupling mechanism is transitionable between a first state, in which, rotation of the proximal driver of the decoupling mechanism advances the overstroke sled with the driver coupling, thereby advancing the drive assembly, and a second state, in which, rotation of the proximal driver disengages the overstroke sled from the driver coupling to enable rotation of the overstroke sled relative to the driver coupling.

12. The surgical instrument according to claim 11, the driver coupling includes a spring in the bore of the driver coupling positioned to bias the overstroke sled towards the slots of the driver coupling.

13. The surgical instrument according to claim 11, wherein the loading unit includes a knife assembly and an actuation sled coupled to the drive assembly of the loading unit for axial displacement with the drive assembly.

14. The surgical instrument according to claim 11, wherein the overstroke sled has a cylindrical body having an outer surface, the bosses diametrically disposed on the outer surface of the cylindrical body.

15. The surgical instrument according to claim 11, wherein the slots of the driver coupling are diametrically arranged and extend along a portion of a length of the driver coupling.

16. The surgical instrument according to claim 11, wherein the tab of the driver coupling extends axially along a portion of a length of the driver coupling.

17. The surgical instrument according to claim 11, wherein the proximal driver, the overstroke sled, and the driver coupling are concentrically arranged.

18. The surgical instrument according to claim 11, wherein the proximal and distal drivers are axially aligned.

19. The surgical instrument according to claim 11, wherein the bosses of the overstroke sled are aligned with the tab of the driver coupling.

20. The surgical instrument according to claim 11, wherein when the drive assembly is in a distal position, rotation of the proximal driver advances the bosses of the overstroke sled through the slots of the driver coupling.
